SEQUENCE LISTING (1 sequence)

SEQ ID NO: 1 - protein, 51 aa, Artificial Sequence
  “epidermal growth factor EGF”; epidermal growth factor EGF
  epidermal growth factor EGF
  epidermal growth factor EGF
NSDSECPLSHDGYCLHDGVCMYIEALDKYACNCVVGYIGERCQYRDLKWWE

(19) United States
(12) Patent Application Publication
(10) Pub. No.: US 2025/0281740 A1
(43) Pub. Date: Sep. 11, 2025

(54) Electroporation process for delivering a composition comprising at least one peptide of molecular weight ranging from 500 Da to 20 kDa

(71) Applicant: L'OREAL, Paris (FR)

(72) Inventors: Patrycja NZOUNZA, Chevilly Larue (FR); Veronique BURNIER, Chevilly Larue (FR)

(21) Appl. No.: 18/564,823

(22) PCT Filed: May 27, 2022
(86) PCT No.: PCT/EP2022/064418

§ 371 Date: Nov. 28, 2023

(30) Foreign Application Priority Data
May 28, 2021 (FR) FR2105587

(51) Int. Cl. A61N 1/32; A61K 8/64; A61N 1/04; A61N 1/36; A61Q 19/08

(52) U.S. Cl. CPC A61N 1/327; A61K 8/64; A61N 1/0416; A61N 1/328; A61N 1/36034; A61Q 19/08

(57) ABSTRACT

Electroporation process for delivering a composition comprising at least one peptide of molecular weight ranging from 500 Da to 20 kDa

The present invention relates to an electroporation process for delivering a composition through human keratin materials, the composition comprising a peptide with a molecular weight ranging from 500 Da to 20 kDa, the electroporation process comprising the exposure of the composition in contact with said keratin materials to a succession of pulses of an electric current, the electric current being supplied by an electroporation device having an electrode (2) and a counterelectrode (3), the pulses each having a duration of between 1 ms and 5 s, with a rest interval between them of between 0.1 s and 5 s, and the electric current having a voltage amplitude ranging from 20 V to 400 V and a current ranging from 0.1 mA to 10 mA.

### CROSS-REFERENCE TO RELATED APPLICATIONS

This application is a U.S. National Stage Entry of International Application No. PCT/EP2022/064418 filed May 27, 2022, which claims the benefit of priority to French Patent Application No. FR2105587 filed May 28, 2021. Each of the foregoing applications is incorporated herein by reference in its entirety.

### SEQUENCE LISTING

The instant application contains a Sequence Listing which has been submitted in ASCII (ST.25) format via the USPTO Patent Center, which is hereby incorporated by reference in its entirety. The ASCII copy, created on Jan. 10, 2024, is named N120678_1030US_PCT_SL.txt and is 859 bytes in size.

### TECHNICAL FIELD

The present invention relates to the field of caring for keratin materials, notably the skin. For the purposes of the present invention, the term “keratin materials” notably denotes the skin, the lips and/or the eyelashes, in particular the skin and/or the lips, and preferably the skin of the body and/or the face, and more preferentially of the face.

Nowadays, a very large number of treatments exist, involving or not involving cosmetic surgery, for enhancing the appearance of human beings. These treatments, which will be referred to hereinbelow as treatments for aesthetic purposes, may act in many ways, and may consist, for example, in effacing or masking certain skin imperfections, or alternatively attenuating or even effacing the signs of ageing.

Skin ageing results from the effects of intrinsic and extrinsic factors on the skin. During the ageing process, a detrimental change in the structure and functions of the skin appears. The main clinical signs due to these modifications of the skin metabolism are the appearance of wrinkles and fine lines, the cause of which is a slackening and loss of the elasticity of the tissues.

Moreover, intrinsic ageing, which brings about the changes of the skin, notably causes a slowing down of the renewal of the cells of the skin, which is reflected essentially by the appearance of detrimental clinical changes, such as the reduction in the subcutaneous adipose tissue and the appearance of small wrinkles or fine lines, and by histopathological changes, such as an increase in the number and thickness of elastic fibres, a loss of vertical fibres from the membrane of the elastic tissue, and the presence of large irregular fibroblasts in the cells of this elastic tissue.

### PRIOR ART

It is known practice to introduce active agents into cosmetic and/or dermatological compositions in order to enhance the appearance of the skin, and notably to combat the signs of ageing.

For example, it has been sought for a long time to formulate peptides, and in particular elastase-inhibiting N-acylamino amide compounds, in the cosmetic and dermatological fields, in various presentation forms, due to their numerous beneficial properties.

In particular, N-acetyl-3-trifluoromethyl-phenyl-(valine-glycine) is a small peptide derivative which has numerous advantageous properties, and which can notably act as an anti-ageing active agent. This peptide increases epidermal regeneration and maintains the epidermal homeostasis.

Thus, on account of their properties, these peptides are efficient for combating the signs of ageing of the skin, for example for improving the radiance of the complexion and fading out wrinkles and/or fine lines in the skin.

Unfortunately, the current formulations do not enable these active agents to penetrate efficiently into keratin materials, and in particular into the skin.

Moreover, it is known that the application of an electric current to the skin can promote the penetration of an active agent. It is thus known practice to treat human keratin materials with the aid of electroporation devices. Electroporation allows the diffusion of active agents through the skin by means of electrical stimulation in a non-invasive manner. Electroporation renders the surface of the skin permeable, allowing active agents to pass by diffusion, or even by electrophoresis when they are charged.

Patent applications US 2010/255079, US 2012/065575, US 2013/345307, US 2013/345661, WO 2010/112708 or WO 2008/045272 describe devices which combine an electric current with cosmetic active agents.

It is also known practice, for example in EP 2 408 801, to use iontophoresis for delivering tetrapeptide elastase inhibitors. Moreover, WO 2010/118880 describes the transdermal application of tripeptides.

Nevertheless, these methods remain, firstly, not very effective, notably for increasing the performance of formulations comprising peptides, and in particular elastase-inhibiting N-acylamino amide compounds, and, secondly, present risks of impairing the skin.

### DISCLOSURE OF THE INVENTION

A need thus remains to further improve the processes which promote the penetration of active agents through keratin materials so as to increase the efficiency of electroporation, and to benefit from a process notably for the cosmetic or dermatological treatment of keratin materials, making it possible to enhance the performance and to accelerate the action of formulations containing peptides with a molecular weight ranging from 500 Da to 20 kDa, and notably elastase-inhibiting N-acylamino amide compounds, in particular N-acetyl-3-trifluoromethyl-phenyl-(valine-glycine), by increasing the supply of active principles inside the live part of the keratin materials, while at the same time being able to be used with comfort and in total safety.

There is in particular interest for home cosmetic treatments that are virtually as efficient and powerful as cosmetic treatments performed by professionals, so as to target deeper levels of the dermis at home and notably to treat moderate to severe wrinkles, damage caused by sunlight and pigmentation, and which give results that become apparent immediately or a short time after a single treatment, painlessly and without any muscle contraction induced by the electroporation.

There is a need to increase the cutaneous release of active agents in a targeted manner inside the live parts of keratin materials, and their bioavailability, so as to have access to efficient home cosmetic treatments.

The invention is directed towards meeting all or some of these needs.

### SUMMARY OF THE INVENTION

Thus, according to a first of its aspects, the present invention relates to an electroporation process for delivering a composition through human keratin materials, the composition comprising at least one peptide with a molecular weight ranging from 500 Da to 20 kDa, and in particular at least one elastase-inhibiting N-acylamino amide compound, the electroporation process involving exposing the composition in contact with said keratin materials to a succession of pulses of an electric current, the electric current being supplied by an electroporation device having at least one electrode and at least one counterelectrode, the pulses each having a duration (tₚᵤₗₛₑ) of between 1 ms and 5 s, better still between 5 ms and 3 s, even better still between 10 ms and 2 s, with a rest interval (T_{off}) between them of between 0.1 s and 5 s, better still between 0.3 s and 3 s, even better still between 0.5 s and 2 s, and the electric current having a voltage amplitude of 20 V to 400 V, better still from 25 V to 300 V, even better still from 30 V to 200 V, and a current ranging from 0.1 mA to 10 mA, better still from 0.2 mA to 2 mA, even better still from 0.25 mA to 1 mA.

The process according to the invention is non-therapeutic and is exclusively for cosmetic purposes.

The term “rest interval” should be understood as meaning the period without pulses which separates two consecutive pulses; preferably, during this interval, no voltage is applied by the electroporation device to the keratin materials.

The number N of consecutive pulses applied may be between 2 and 15 000, better still between 10 and 5000, and even better still between 50 and 500. These N pulses which follow each other are referred to hereinbelow as the “pulse train”.

Preferably, the polarity of the voltage applied is constant, being chosen as a function of that of the compound(s) whose diffusion in the keratin materials it is desired to improve.

The process according to the invention makes it possible to perform home cosmetic treatments that are much more efficient than the conventional home cosmetic treatments, with results which become apparent immediately or a short time after a single treatment. Indeed, the cutaneous release of peptides with a molecular weight ranging from 500 Da to 20 kDa, and in particular of elastase-inhibiting N-acylamino amide compounds, into the live parts of the keratin materials, and also their bioavailability, are increased, in comparison with conventional home cosmetic treatments, in a painless manner, without muscular contraction induced by the electroporation and without any impairment of the keratin materials, and in particular of the skin.

In particular, the process according to the invention allows changes in skin impedances and thus an increase in the permeability of peptides with a molecular weight ranging from 500 Da to 20 kDa, and in particular of elastase-inhibiting N-acylamino amide compounds, through keratin materials, and notably through the skin.

Thus, the diffusion of peptides with a molecular weight ranging from 500 Da to 20 kDa, and in particular of elastase-inhibiting N-acylamino amide compounds, through keratin materials, and notably through the skin, is increased by applying the process according to the invention.

According to a preferred embodiment, the process comprises topical application of the composition to the zone to be treated before applying the electric current and/or during said application.

### DETAILED DESCRIPTION

### Electroporation Device

According to the invention, the electric current is provided by an electroporation device having at least one electrode and at least one counterelectrode.

According to the invention, the pulses preferably have a voltage amplitude ranging from 30 V to 200 V, and a current ranging from 0.25 mA to 1 mA. The amplitude is measured peak-to-peak and the current is the instantaneous current.

The maximum current density per pulse may be less than or equal to 0.5 mA/cm².

Preferably, the electric current has a direct polarity (“DC current”), the polarity of the electrode being, for example, negative or positive with respect to that of the counterelectrode, depending on the polarity of the applied compounds. Preferably, the polarity of the electrode is negative with respect to that of the counterelectrode.

The pulses may have a constant maximum amplitude. The pulses may have, from one pulse to the next, a variable amplitude, for example an amplitude which increases from a minimum amplitude, reaches the maximum amplitude, and then eventually decreases towards the minimum amplitude. It is possible to combine two or more pulse waveforms simultaneously or alternately. The application of the current may involve the generation of a current having sinusoidal waveforms, non-sinusoidal waveforms, periodic square waveforms, rectangular waveforms, sawtooth waveforms, spike waveforms, bell waveforms, trapezoidal waveforms, triangular waveforms, or combinations thereof.

Preferably, the electric current is of square or rectangular waveform. A pulse may thus have a square-wave voltage shape.

According to a preferred embodiment, the applied current has a duty cycle (tₚᵤₗₛₑ/(tₚᵤₗₛₑ+T_{off})) ranging from 10% to 90%, preferably from 20% to 80%, where tₚᵤₗₛₑ denotes the duration of a pulse and T_{off} denotes the interval between two successive pulses.

Preferably, the maximum voltage between the electrode and the counterelectrode in open circuit ranges from 150 V to 250 V.

### Electrodes

According to a preferred embodiment, the process comprises the step consisting in manually moving the electrode(s) of the electroporation device over the keratin materials while subjecting said keratin materials to said pulses of electric current.

According to another embodiment, the active electrode is stationary and applied at a single location on the keratin materials, for example through an adhesive.

The electrode(s) may have a rounded surface for contact with the keratin materials.

Preferably, the surface area of the electrode(s) in contact with the keratin materials ranges from 2 cm² to 20 cm². The current density ranges, for example, from 0.1 mA/cm² to 0.5 mA/cm². Preferably, the counterelectrode is static and has a surface area in contact with the human keratin materials that is greater than that of the electrode, which is moved in contact with the keratin materials, the surface area of the counterelectrode preferably being more than 1.5 times that of the electrode.

The electroporation device may comprise at least one reservoir containing an effective amount of a composition according to the invention.

In particular, the device may comprise at least one electrode and one or more compositions stored in a reservoir such as a cavity, a gel, a laminate, a membrane, a porous structure, a matrix or a substrate.

More particularly, the device may comprise at least one active electrode electrically coupled to a reservoir containing a cosmetic composition according to the invention.

The reservoir may comprise any shape or material used for retaining a composition according to the invention.

The reservoir may comprise one or more ion-exchange membranes, semi-permeable membranes, porous membranes or gels that are capable of at least temporarily retaining a composition according to the invention.

The reservoir may comprise one or more cavities formed by a structure.

In particular, the electroporation device may comprise one or more ion-exchange membranes that may be positioned to serve as a polarity-selective barrier between the reservoir of composition according to the invention and a biological interface.

The reservoir may be a hollow imprint at the end of the electrode. As a variant, according to a particular embodiment, the reservoir is an absorbent material for containing the composition according to the invention.

In particular, the reservoir may be connected to a piston or to any other means for dispensing the composition. Thus, the active electrode may be provided with a type of ball applicator fed by a piston. The piston may be actuated during use of the electroporation device.

The electroporation device may also be an electroporation patch or an electroporation face mask.

Preferably, the electroporation device is hand-held.

### User Interface

The electroporation device may comprise a user interface.

The user interface may be intended for entering data regarding the types of waveforms to be applied as electrical stimuli. The user interface may comprise an alphanumeric keyboard and a display. The alphanumeric keyboard may be implemented as a touch screen.

In particular, the user interface may comprise directional arrow buttons and an input button for entering data into a memory.

The input of waveform parameters can be done by using text boxes and/or by using drop-down menus. Irrespective of how data is entered, the user interface may communicate a variety of prompts for the user to enter information.

The user interface may prompt the user to enter the duration of the treatment.

The user interface may prompt the user to enter the treatment zone.

The device may include a controller which runs logic routines to direct the user interface so as to present the user with appropriate prompts. The controller then uses the entered parameters to generate, via its electronics, including, but not limited to, the appropriate voltage based on the parameters entered by the user.

### Other Sensors

According to a particular embodiment of the electroporation process, the process comprises a step of measuring the skin temperature, the skin impedance and the pH of the composition. Thus, the electroporation device may comprise at least a temperature sensor, an impedance sensor, and/or a pH sensor.

The application of the current profile can thus be reduced to a safety level when a value measured by one of the sensors exceeds a safety range or value.

According to a particular embodiment of the electroporation process, the process comprises a step of measuring the pH of the composition. When the measured pH exceeds a pH safety range, the application of the current profile is switched to a safety level, for example a safety level below 1 V, such as 0.5 V. The pH safety range is, for example, from pH 4 to 7. In certain embodiments, when the measured pH exceeds a pH safety range, for example the range from 4 to 7, the device switches the polarity for a short period of time to allow the pH to be reequilibrated.

According to a particular embodiment of the electroporation process, the process comprises a step of measuring the impedance of the skin. When the measured impedance exceeds an impedance safety range, the application of the current profile is reduced to a safety level to avoid an adverse event. The safety level may be less than 1 V, such as 0.5 V. The impedance safety range may be from 5 kΩ to 2 MΩ.

According to a particular embodiment of the electroporation process, the process comprises a step of measuring the temperature of the skin. When the measured temperature exceeds a temperature safety value, the application of the current profile is switched to a safety level, for example less than 1 V, such as 0.5 V. The temperature safety value can be chosen to be less than or equal to 43° C.

In a preferred embodiment of the electroporation process, the process comprises the steps of:
measuring the temperature of the skin,
measuring the impedance of the composition, and
measuring the pH of the composition.

The device is configured to process the results and to regulate the microcurrent and the polarity.

### Process

The process according to the invention may be cosmetic and non-therapeutic.

According to a particular embodiment of the electroporation process, the process comprises the transdermal delivery of a composition according to the invention, including one or more peptides with a molecular weight ranging from 500 Da to 20 kDa, and in particular one or more elastase-inhibiting N-acylamino amide compounds.

According to a particular embodiment of the invention, the process for delivering the composition according to the invention comprising at least one peptide with a molecular weight ranging from 500 Da to 20 kDa, notably at least one elastase-inhibiting N-acylamino amide compound, in particular N-acetyl-3-trifluoromethyl-phenyl-(valine-glycine), through the keratin materials, involves applying a specific electric current profile using an electroporation device containing at least one electrode and at least one counterelectrode, for a specific time sufficient to transdermally deliver the peptide(s) with a molecular weight ranging from 500 Da to 20 kDa, notably the elastase-inhibiting N-acylamino amide compound(s), in particular N-acetyl-3-trifluoromethyl-phenyl-(valine-glycine), to a biological subject.

Thus, the diffusion through human keratin materials of the peptide(s) with a molecular weight ranging from 500 Da to 20 kDa, notably of the elastase-inhibiting N-acylamino amide compound(s), in particular of N-acetyl-3-trifluoromethyl-phenyl-(valine-glycine), can be increased by applying the electroporation process according to the invention. Such an increase in the diffusion of the active agent makes it possible to optimize the amount of active agent required for the target treatments in the various layers of the keratin materials. The electroporation treatment can be applied for a period of 60 s to 1000 s.

In particular, the electroporation treatment is applied for a period of 120 s to 480 s.

### Kit

According to a particular embodiment, the invention also proposes an electroporation kit comprising:
an electroporation composition comprising at least one peptide with a molecular weight ranging from 500 Da to 20 kDa, notably at least one elastase-inhibiting N-acylamino amide compound, in particular N-acetyl-3-trifluoromethyl-phenyl-(valine-glycine), and
an electroporation device for performing the electroporation process as described above.

The present invention also proposes an electroporation kit comprising:
an electroporation composition comprising at least one peptide with a molecular weight ranging from 500 Da to 20 kDa, notably at least one elastase-inhibiting N-acylamino amide compound, in particular N-acetyl-3-trifluoromethyl-phenyl-(valine-glycine), and
an electroporation device for delivering the electroporation composition through keratin materials, which is configured to apply a negative current profile.

Preferably, the present invention proposes a kit for performing the process according to the invention, including:
an electroporation device containing at least one electrode (2) and at least one counterelectrode (3) and arranged to subject the keratin materials to a succession of pulses of an electric current, the pulses each having a duration (tₚᵤₗₛₑ) of between 1 ms and 5 s, better still between 5 ms and 3 s, even better still between 10 ms and 2 s, with a rest interval (T_{off}) between them of between 0.1 s and 5 s, better still between 0.3 s and 3 s, even better still between 0.5 s and 2 s, and the electric current having a voltage amplitude ranging from 20 V to 400 V, better still from 25 V to 300 V, even better still from 30 V to 200 V, and a current ranging from 0.1 mA to 10 mA, better still from 0.2 mA to 2 mA, even better still from 0.25 mA to 1 mA, and
a composition comprising at least one peptide with a molecular weight ranging from 500 Da to 20 kDa, notably at least one elastase-inhibiting N-acylamino amide compound, in particular at least N-acetyl-3-trifluoromethyl-phenyl-(valine-glycine).

The electroporation device and the composition may be packaged together, for example in the same package.

### Applications

In a preferred embodiment, the process, the composition and the kit according to the invention enable the treatment of wrinkles and the signs of ageing, and improve the softness, quality and appearance of the skin. It can thus be used to moisturize the skin, to improve the suppleness of the skin, to improve and/or reduce the microrelief of the skin, and also to combat the signs of skin ageing.

The process, the composition and the kit according to the invention are notably effective in delivering anti-ageing ingredients which impregnate the skin beyond the superficial levels, which may contribute to such effects. After ablative laser procedures or chemical peeling operations, for example, electroporation can also improve the healing process by achieving higher delivery of active agents. The rapid penetration that reaches a greater depth makes electroporation also effective for acne scars.

According to another particular embodiment, the process is used for minimizing the effects of age on the skin, and/or pigmentation, and/or the volume and/or sagging of wrinkles, and/or tone and/or spots, and/or for improving the firmness, and/or radiance, and/or softness of the skin.

### DESCRIPTION OF THE FIGURES

Other characteristics, variants and advantages of the invention will become apparent on reading the description of non-limiting illustrative examples thereof and on examining the appended drawings, in which:

### FIGURES

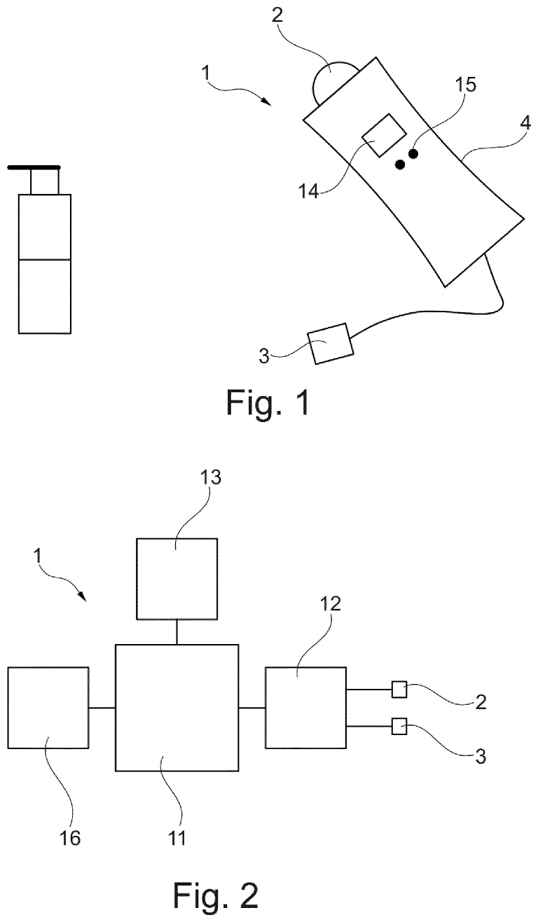

FIG. 1 is a schematic representation of an example of an electroporation device according to the invention,

FIG. 2 is a block diagram of the device,

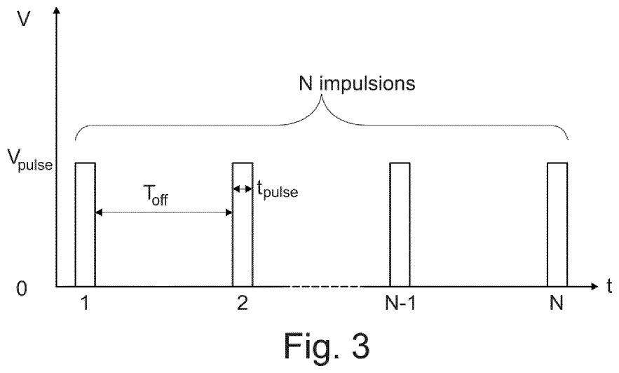

FIG. 3 illustrates an example of an applied voltage waveform,

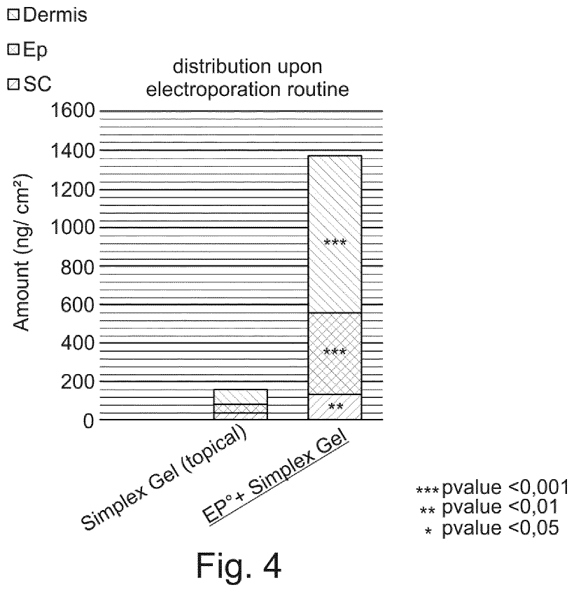

FIG. 4 is a diagram comparing the delivery of N-acetyl-3-trifluoromethyl-phenyl-(valine-glycine) from a 1% simplex N-acetyl-3-trifluoromethyl-phenyl-(valine-glycine) formulation topically into keratin materials and the delivery of N-acetyl-3-trifluoromethyl-phenyl-(valine-glycine) from a 1% simplex N-acetyl-3-trifluoromethyl-phenyl-(valine-glycine) formulation by electroporation, and

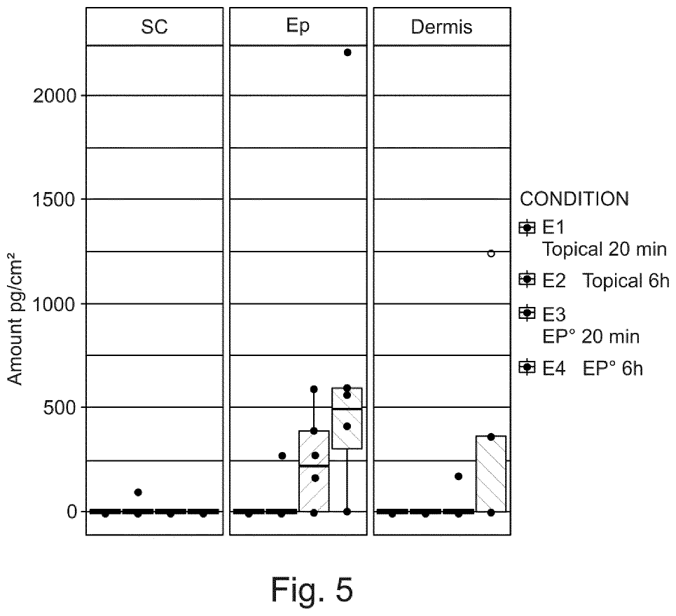

FIG. 5 represents the amount of EGF that has penetrated into the skin by means of an ELISA technique.

FIG. 1 shows an example of a treatment device 1 according to the invention.

This device 1 has at least two electrical outputs between which an electrical signal is generated.

In the example considered, the device 1 includes at least one electrode 2, to be moved over the skin in the zones to be treated, and a counterelectrode 3 to be fixed onto the body, for example by means of a bracelet or an adhesive patch.

The electrode 2 can be carried, as shown in FIG. 1, by a case 4 which houses an electronic signal-generating circuit or, in a variant, not shown, it is carried by a handpiece which is connected via a cable to a static box housing the electronic signal-generating circuit.

The electronic signal-generating circuit may include, as illustrated in FIG. 2, a central unit 11 with a microcontroller, for example of the ATMEGA type, which runs software for generating a signal of the desired shape, which is amplified by an amplifier 12 connected to the electrode 2 and to the counterelectrode 3.

The central unit 11 also manages the user interface 13, which includes, for example, a display 14 and one or more control buttons 15.

The electronic circuit can be powered by a battery 16 housed in the case 4, or as a variant by a mains adapter.

The output signal can be modulated.

The output signal may be a periodic signal, of given elemental pattern, which is modulated. For example, as shown in FIG. 3, the applied voltage is periodic, with N square-wave pulses emitted, each with a duration tₚᵤₗₛₑ of 10 ms, for example, separated by pulse-free rest periods T_{off} with a duration equal to 500 ms, for example.

A duration tₚᵤₗₛₑ of less than or equal to 10 ms can reduce the risk of skin irritation or pain sensation or induction of muscle contractions, given that the nature of the applied field, in particular the geometry and configuration of the electrodes (very close or very far apart, superficial versus through electric field when the electrodes are far apart), can lead to different perception thresholds.

A rest period T_{off} of greater than or equal to 100 ms allows the skin to recover a basal membrane potential between the application of the pulses.

The device 1 can be configured to generate an electrical signal between the electrode and the counterelectrode, for a predefined number of pulses or of pulse trains, for example by means of a pulse counter, or for a predefined duration, by means of a timer.

The electrode 2 preferably has a rounded shape.

The skin contact area of the electrode 2 ranges, for example, from 2 cm² to 20 cm². The electrical current density applied by the electrode 2 ranges, for example, from 0.1 mA/cm² to 0.5 mA/cm²

The voltage between the electrode 2 and the counterelectrode 3 preferably increases when a resistive load is connected thereto and when the resistance of the load increases. The amplitude is, for example, substantially zero for a zero resistance up to a resistance of 10 kOhms, then increases until it reaches about 200 V in open circuit.

The current (RMS) delivered by the device is preferably between 150 mA and 500 mA when it flows over a resistor whose value varies between 1 kOhm and 500 kOhms.

The device can be offered to the user with a composition P according to the invention to be applied to the skin, inside a common packaging, for instance a box.

The composition P according to the invention may be contained in any type of container, for example a container equipped with an applicator for applying the composition to the skin, or a pump bottle, among other possibilities.

Preferably, the applied voltage is unipolar, the voltage of the electrode 2 being, for example, either always positive or always negative, depending on the nature of the active agent(s) contained in the composition.

### Composition

An electroporation composition for use in an electroporation method as mentioned above is described.

A composition according to the invention has a viscosity such that the composition covers both the treated surface and the treatment electrode of the apparatus, allowing smooth, frictionless movement.

A composition according to the invention preferably has a viscosity of between 0.001 Pa·s and 0.5 Pa·s, better still between 0.005 Pa·s and 0.1 Pa·s, and even better still between 0.01 Pa·s and 0.05 Pa·s.

The viscosity of the composition is measured at room temperature (25° C.) using an RM 200 Plus rheometer (LamyRheology).

According to a first preferred embodiment, a composition according to the invention is of gel type, in particular of aqueous gel type.

According to another preferred embodiment, a composition according to the invention is an emulsion, notably a water-in-oil or oil-in-water emulsion, and preferably an oil-in-water emulsion, also known as a direct emulsion.

Preferably, a composition according to the invention is of gel type, or is an oil-in-water emulsion.

Thus, a composition according to the invention may comprise an aqueous phase and/or an oily phase.

In certain embodiments of the electroporation composition, the composition has a pH ranging from 2 to 7.5, preferably from 3 to 6, and more preferentially from 3 to 5.

In certain embodiments, when the measured pH exceeds a pH safety range, for example the range from 4 to 6, the device switches the polarity for a short period of time to allow the pH to reequilibrate.

As mentioned above, the electroporation method according to the present invention comprises a step of applying to the skin a composition comprising a peptide with a molecular weight ranging from 500 Da to 20 kDa, in particular an N-acylamino amide compound having inhibitory activity on the activity of elastases.

Peptides with a Molecular Weight Ranging from 500 Da to 20 kDa

Peptides are polymers of amino acids linked via peptide bonds.

According to the invention, the peptides used in the composition have a molecular weight ranging from 500 Da to 20 kDa.

According to the invention, the molecular weight may be measured, for example, by mass spectrometry or by gel filtration chromatography.

According to a preferred embodiment of the invention, the peptides have a molecular weight ranging from 100 Da to 50 kDa, and more preferentially from 500 Da to 20 kDa.

The peptides of the invention may be modified by acylation on their N-terminal function and/or by esterification on their C-terminal function.

The peptides of the invention may be obtained by chemical or enzymatic synthesis from the constituent amino acids or derivatives thereof.

Finally, the peptides of the invention may be obtained by biotechnology (use of a microorganism optionally modified by genetic engineering).

A person skilled in the art, knowing the field of extraction and purification of peptides and proteins, can easily envisage other processes for obtaining the peptides used in the composition.

The peptides used in the composition may notably be chosen from an EGF (epidermal growth factor), VEGF (vascular endothelial growth factor), FGF (fibroblast growth factor) or PDGF (platelet-derived growth factor) peptide, or elastase-inhibiting N-acylamino amide compounds.

Preferably, the peptides with a molecular weight ranging from 500 Da to 20 kDa are chosen from elastase-inhibiting N-acylamino amide compounds and/or epidermal growth factor EGF of sequence SEQ ID No. 1.

According to a preferred embodiment, the peptides with a molecular weight ranging from 500 Da to 20 kDa are chosen from elastase-inhibiting N-acylamino amide compounds. The elastase-inhibiting N-acylamino-amide compound according to the present invention may notably be a compound of formula (I) below:

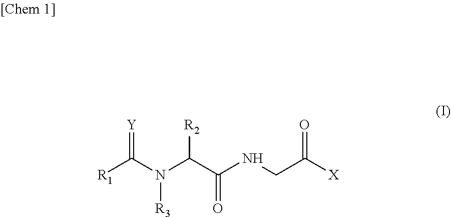

in which:
   the radical Y represents 0 or S;
   the radical Ri represents:
(i) a hydrogen atom;
(ii) a linear, branched or cyclic, saturated or unsaturated hydrocarbon-based radical containing from 1 to 18 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; and —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; —C-Hal₃ (halogen), in particular —CF₃; with R″ representing a linear, branched or cyclic, saturated or unsaturated hydrocarbon-based radical containing 1 to 6 carbon atoms, which is optionally halogenated, or even perhalogenated;
(iii) a radical chosen from the radicals —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
   the radical R₂ represents a linear, branched or cyclic, saturated or unsaturated hydrocarbon-based radical containing 1 to 18 carbon atoms, optionally substituted with 1 to 5 identical or different groups chosen from —OH; —OR; —O—COR; —SH; —SR; —S—COR; —NH₂; —NHR; —NRR″; —NH—COR; -Hal (halogen); —CN; —COOR; and —COR; with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
   the radical R₃ represents a radical chosen from those of formula (II) or (III):

-A-C₆H_{(5-y)}—B_{y} (II)

—C₆H_{(5-y)}—B_{y′} (III)
in which:
   y is an integer between 0 and 5 inclusive;
   y′ is an integer between 0 and 5 inclusive;
   A is a divalent, linear, branched or cyclic, saturated or unsaturated, hydrocarbon-based radical containing 1 to 18 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; and —SO₂—OR; with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
   B is a divalent, linear, branched or cyclic, saturated or unsaturated hydrocarbon-based radical containing 1 to 18 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen, or even perhalogen); —C-Hal₃ (halogen), in particular —CF₃; —CN; —COOR; —COR; —NO₂; and —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated;
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
   the radical X represents a radical chosen from —OH; —OR₄; —NH₂; —NHR₄; —NR₄R₅; —SR₄; —COOR₄; and —COR₄;
with R₄ and R₅ representing, independently of each other, a linear, branched or cyclic, saturated or unsaturated hydrocarbon-based radical containing 1 to 6 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen, or even perhalogen); —CN; —COOR; and —COR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
it being possible for said radicals R₄ and R₅ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
mineral or organic acid salts thereof, optical isomers thereof, in isolated form or as a racemic mixture.

Preferably, the radical Y represents 0 and/or the radical X represents —OH.

According to a preferred embodiment, -Hal (halogen) is fluorine.

The term “linear, branched or cyclic hydrocarbon-based radical” notably means radicals of alkyl, aryl, aralkyl, alkylaryl, alkenyl and alkynyl type.

The C₆H₅ group present in the radical R₃ should be included as an aromatic cyclic group.

Preferably, the radical Y represents an oxygen atom.

Preferably, the radical Ri represents a hydrogen atom or an optionally substituted linear, branched or cyclic, saturated or unsaturated hydrocarbon-based radical containing 1 to 12, and notably 1, 2, 3, 4, 5 or 6 carbon atoms.

In particular, the substituents may be chosen from —OH, —OR and/or —P(O)—(OR)₂ with R representing a linear, branched or cyclic, saturated or unsaturated hydrocarbon-based radical containing 1 to 6 carbon atoms, which is optionally halogenated, or even perhalogenated.

Preferably, the radical Ri represents a methyl, ethyl, propyl or isopropyl radical, optionally substituted with an —OH or —P(O)—(OR)₂ group with R representing a methyl, ethyl, propyl or isopropyl radical.

Preferably, the radical R₂ represents an optionally substituted linear, branched or cyclic, saturated or unsaturated hydrocarbon-based radical containing 1 to 12, and in particular 1, 2, 3, 4, 5 or 6 carbon atoms.

In particular, the substituents may be chosen from —OH and —OR with R representing a linear, branched or cyclic, saturated or unsaturated hydrocarbon-based radical containing 1 to 6 carbon atoms, which is optionally halogenated, or even perhalogenated.

Preferably, the radical R₂ represents a methyl, ethyl, propyl, isopropyl, n-butyl, tert-butyl or isobutyl radical.

Preferably, the radical R₃ represents a radical of formula —C₆H_{(5-y′)}—B_{y′}, in which y′ =1, 2 or 3; or a radical of formula -A-C₆H_{(5-y)}—B_{y}, in which y=0, 1 or 2. Preferably, A is an optionally substituted, linear, branched or cyclic, saturated or unsaturated divalent hydrocarbon-based radical containing 1 to 12 carbon atoms. The substituents of A are preferably chosen from -Hal (halogen, or even perhalogen); —CN; —COOR; —NO₂; —SO₂—OR; with R representing a linear, branched or cyclic, saturated or unsaturated hydrocarbon-based radical containing from 1 to 6 carbon atoms, which is optionally halogenated, or even perhalogenated. Preferably, the radical R₃ represents a group chosen from one of the formulae below:

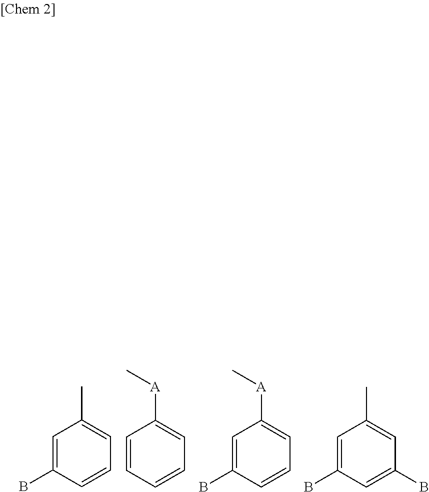

in which A and B have the meanings given above.

In particular, the divalent radical A may be methylene, ethylene or propylene.

The radical B is preferably a methyl, ethyl, propyl or isopropyl radical, substituted with one or more halogens, in particular chlorine, bromine, iodine or fluorine, and preferably totally halogenated (perhalogenated), for instance perfluorinated. Mention may be made in particular of the perfluoromethyl radical (—CF₃) as being most particularly preferred.

Preferably, the radical X represents a radical chosen from —OH and —OR₄ with R₄ representing an optionally substituted linear, cyclic or branched, saturated or unsaturated hydrocarbon-based radical containing 1 to 6 carbon atoms. The substituents may be chosen from —OH and —OR with R representing a linear, branched or cyclic, saturated or unsaturated hydrocarbon-based radical containing 1 to 6 carbon atoms, which is optionally halogenated, or even perhalogenated. Preferably, the radical X represents a radical chosen from —OH, —OCH₃, —OC₂H₅, —O—C₃H₇ or —OC₄H₉.

Preferably, the elastase-inhibiting N-acylamino amide compound is at least N-acetyl-3-trifluoromethyl-phenyl-(valine-glycine).

In the context of the present invention, the term “N-acetyl-3-trifluoromethyl-phenyl-(valine-glycine)” notably covers the basic unit having the following formula and the derivative thereof:

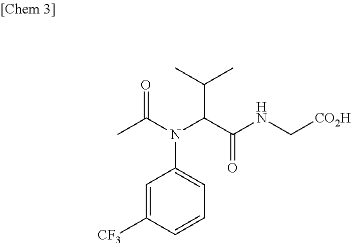 [SMILES: CC(=O)N(c1cccc(C(F)(F)F)c1)C(C(=O)NCC(=O)O)C(C)C]

In the context of the present invention, the term “N-acetyl-3-trifluoromethyl-phenyl-(valine-glycine) or a derivative thereof” also includes N-acetyl-3-trifluoromethyl-phenyl-(valine-glycine) salts, and in particular alkali metal salts such as the sodium salt and the potassium salt.

N-Acetyl-3-trifluoromethyl-phenyl-(valine-glycine) is an anti-ageing agent.

According to embodiments of the electrical method, the method also comprises the transdermal delivery of a composition comprising a peptide with a molecular weight ranging from 500 Da to 20 kDa, notably an elastase-inhibiting N-acylamino amide compound, in particular one or more from among N-acetyl-3-trifluoromethyl-phenyl-(valine-glycine), N-acetyl-3-trifluoromethyl-phenyl-(valine-glycine) derivatives, N-acetyl-3-trifluoromethyl-phenyl-(valine-glycine) ions, and ions of N-acetyl-3-trifluoromethyl-phenyl-(valine-glycine) derivatives, present in an amount ranging from 0.1% to 20% by weight.

The peptide with a molecular weight ranging from 500 Da to 20 kDa, notably the elastase-inhibiting N-acylamino amide compound, in particular N-acetyl-3-trifluoromethyl-phenyl-(valine-glycine) or a derivative thereof, may be present in the composition according to the present invention in a content of between 0.2% and 10%, preferably between 0.5% and 5%, and more particularly between 0.8% and 2% by weight, relative to the total weight of the composition.

In one embodiment, the composition comprises 1% by weight of at least one peptide with a molecular weight ranging from 500 Da to 20 kDa, notably at least one elastase-inhibiting N-acylamino amide compound, and in particular N-acetyl-3-trifluoromethyl-phenyl-(valine-glycine).

The compounds according to the invention may be readily prepared by a person skilled in the art on the basis of his general knowledge. In particular, a carboxylic acid, an aldehyde, an amino compound and an isonitrile can be reacted according to the Ugi reaction.

Needless to say, during the synthesis of the compounds according to the invention, and depending on the nature of the various radicals present in the starting compounds, a skilled person will take care to protect certain substituents so that they do not react in the course of the reactions.

According to another preferred embodiment, the peptide with a molecular weight ranging from 500 Da to 20 kDa is at least the epidermal growth factor EGF of sequence SEQ ID No. 1.

According to embodiments of the process, the process also comprises the transdermal delivery of a composition comprising the epidermal growth factor EGF of sequence SEQ ID No. 1, present in an amount ranging from 0.2% to 10% by weight.

The epidermal growth factor EGF of sequence SEQ ID No. 1 may be present in the composition according to the present invention in a content of between 0.2% and 10%, preferably between 0.5% and 5% and more particularly between 0.8% and 2% by weight relative to the total weight of the composition.

In one embodiment, the composition comprises 1% by weight of epidermal growth factor EGF of sequence SEQ ID No. 1.

### Aqueous Phase

A composition according to the invention preferably comprises an aqueous phase.

According to a preferred embodiment, the composition according to the present invention has a water content of between 20% and 100% by weight, in particular between 40% and 95% by weight and preferably between 60% and 90% by weight relative to the total weight of the composition.

According to a preferred embodiment, the aqueous phase of a composition according to the invention also comprises at least one alcohol, notably chosen from polyols, in particular chosen from glycols.

Thus, the composition may also comprise a polyol chosen from propylene glycol, 1,3-propanediol, dipropylene glycol, butylene glycol, pentylene glycol, hexylene glycol, caprylyl glycol, glycerol and sugars such as sorbitol, (poly)alkylene glycols, and mixtures thereof.

Preferably, the composition may comprise a polyol chosen from dipropylene glycol, caprylyl glycol and glycerol, and mixtures thereof.

According to one embodiment, the mass concentrations of polyol range from 0.01% to 40% by weight, relative to the total weight of said composition.

Preferably, the mass concentrations of polyol range from 0.1% to 30% by weight and preferably from 5% to 15% by weight relative to the total weight of said composition.

### Thickener/Gelling Agent

According to preferred embodiments, a composition according to the present invention may comprise a thickener/gelling agent.

Thus, depending on the viscosity of the composition that it is desired to obtain, one or more thickeners/gelling agents, which are notably hydrophilic, i.e. soluble or water-dispersible, may be incorporated into the composition.

Examples of hydrophilic gelling agents that may be mentioned include modified or unmodified carboxyvinyl polymers, such as the products sold under the names Carbopol (CTFA name: Carbomer) and Pemulen (CTFA name: Acrylates/C₁₀₋₃₀ alkyl acrylate crosspolymer) by the company Goodrich, polyacrylamides, optionally crosslinked and/or neutralized 2-acrylamido-2-methylpropanesulfonic acid polymers and copolymers, for instance the poly(2-acrylamido-2-methylpropanesulfonic acid) sold by the company Hoechst under the name Hostacerin AMPS® (CTFA name: Ammonium polyacryldimethyltauramide), crosslinked anionic copolymers of acrylamide and of AMPS®, which are in the form of a water-in-oil emulsion, such as those sold under the name Sepigel 305 (CTFA name: Polyacrylamide/C₁₃₋₁₄ Isoparaffin/Laureth-7) and under the name Simulgel 600 (CTFA name: Acrylamide/Sodium acryloyldimethyltaurate copolymer/isohexadecane/Polysorbate 80) by the company SEPPIC, polysaccharide biopolymers such as modified celluloses, carrageenans, notably kappa-carrageenan, gellan gum, agar-agar, xanthan gum, alginate-based compounds, in particular sodium alginate, scleroglucan gum, guar gum, inulin and pullulan, cassia gum, karaya gum, konjac gum, gum tragacanth, tara gum, acacia gum or gum arabic, and mixtures thereof. The amount of gelling agents depends on the desired objective.

According to a preferred embodiment, a composition according to the present invention comprises a polysaccharide, notably chosen from a cellulose or a derivative thereof, in particular cellulose ethers or esters.

The cellulose derivatives may be anionic, cationic, amphoteric or nonionic. Among these derivatives, cellulose ethers, cellulose esters and cellulose ether esters are distinguished.

Among the nonionic cellulose ethers, mention may notably be made of alkylcelluloses such as methylcelluloses and ethylcelluloses, hydroxyalkylcelluloses such as hydroxymethylcelluloses, hydroxyethylcelluloses and hydroxypropylcelluloses, and mixed hydroxyalkylalkylcelluloses such as hydroxypropylmethylcelluloses, hydroxyethylmethylcelluloses, hydroxyethylethylcelluloses and hydroxybutylmethylcelluloses.

The celluloses and derivatives are represented, for example, by the products sold under the names Avicel® (microcrystalline cellulose, MCC) by the company FMC Biopolymers, under the name Cekol (carboxymethylcellulose) by the company Noviant (CP-Kelco), under the name Akucell AF (sodium carboxymethylcellulose) by the company Akzo Nobel, under the name Methocel™ (cellulose ethers) and Ethocel™ (ethylcellulose) by the company Dow, and under the names Aqualon® (carboxymethylcellulose and sodium carboxymethylcellulose), Benecel® (methylcellulose), Blanose™ (carboxymethylcellulose), Culminal® (methylcellulose, hydroxypropylmethylcellulose), Klucel® (hydroxypropylcellulose), Polysurf® (cetylhydroxyethylcellulose) and Natrosol® CS (hydroxyethylcellulose) by the company Hercules Aqualon.

According to a preferred embodiment, a composition according to the invention comprises at least one hydroxypropylmethylcellulose and/or a crosslinked anionic copolymer of acrylamide and of AMPS®.

According to a preferred embodiment, a composition according to the invention comprises at least one hydroxypropylmethylcellulose.

According to a preferred embodiment, a composition according to the invention comprises at least one crosslinked anionic copolymer of acrylamide and of AMPS®.

In particular, the amount of thickeners/gelling agents ranges, for example, from 0.01% to 10%, for example from 0.1% to 5% by weight, relative to the total weight of the composition.

### Oily Phase

As mentioned above, a composition according to the invention may comprise an oily phase. When the composition used according to the invention comprises an oily phase, it preferably contains at least one oil, notably a cosmetic oil. It may also contain other fatty substances. Among the oils that may be used in the composition of the invention, mention may be made of hydrocarbon-based oils of animal origin, hydrocarbon-based oils of plant origin, synthetic esters and ethers, notably of fatty acids, hydroxylated esters, polyol esters, pentaerythritol esters, linear or branched hydrocarbons of plant, mineral or synthetic origin, fatty alcohols and fatty acids containing from 8 to 26 carbon atoms, silicone-based and/or partially hydrogenated fluoro oils, silicone oils and mixtures thereof.

According to a preferred embodiment, a composition according to the invention comprises at least one silicone oil.

The silicone oils may be chosen, for example, from volatile or non-volatile, linear or cyclic polydimethylsiloxanes (PDMS) which are liquid or pasty at room temperature, notably cyclopolydimethylsiloxanes (cyclomethicones) such as cyclohexasiloxane; polydimethylsiloxanes including alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyltrimethyl siloxysilicates; and polymethylphenylsiloxanes.

According to a preferred embodiment, a composition according to the invention comprises at least one cyclohexasiloxane oil.

The other fatty substances that may be present in the oily phase are, for example, fatty acids comprising from 8 to 30 carbon atoms, for example stearic acid, lauric acid, palmitic acid and oleic acid; waxes, for example lanolin wax, beeswax, carnauba wax or candelilla wax, paraffin wax, lignite wax or microcrystalline waxes, ceresin or ozokerite, and synthetic waxes, for example polyethylene waxes and Fischer-Tropsch waxes; silicone resins such as trifluoromethyl (C₁-C₄)alkyl dimethicone and trifluoropropyl dimethicone; and silicone elastomers.

These fatty substances may be chosen in a varied manner by a person skilled in the art in order to prepare a composition having the desired properties, for example in terms of consistency or texture.

According to a preferred embodiment, a composition according to the invention comprises at least one silicone elastomer.

The term “silicone elastomer” or “organopolysiloxane elastomer” means a soft, deformable organopolysiloxane with viscoelastic properties and notably with the consistency of a sponge or soft sphere. Its modulus of elasticity is such that this material withstands deformation and has limited stretchability and contractility. This material is capable of regaining its original shape after stretching.

It is more particularly a crosslinked organopolysiloxane elastomer.

In particular, the silicone elastomer used in the present invention is chosen from Dimethicone Crosspolymer (INCI name), Vinyl Dimethicone Crosspolymer (INCI name), Dimethicone/Vinyl Dimethicone Crosspolymer (INCI name), Dimethicone Crosspolymer-3 (INCI name).

According to a preferred embodiment, the composition according to the invention comprises at least one crosslinked silicone elastomer having the INCI name “dimethicone crosspolymer” or “dimethicone (and) dimethicone crosspolymer”, with, preferably, a dimethicone having a viscosity ranging from 1 to 100 cSt, in particular from 1 to 10 cSt at 25° C., such as the mixture of polydimethylsiloxane crosslinked with Hexadiene/Polydimethylsiloxane (5 cSt) sold under the name DC 9041© by the company Dow Corning or the mixture of polydimethylsiloxane crosslinked with Hexadiene/Polydimethylsiloxane (2 cSt) sold under the name EL-9240® by the company Dow Corning.

The silicone elastomer may be present in a composition according to the invention in a content of between 0.1% and 20% by weight, notably between 0.5% and 10% by weight and more particularly between 1% and 5% by weight relative to the total weight of the composition.

In particular, the amount of oily phase may range, for example, from 0% to 30%, and more particularly from 0.1% to 15% by weight, relative to the total weight of the composition.

### Surfactant/Emulsifier

According to one embodiment, the composition according to the invention may comprise at least one emulsifier.

Among the emulsifiers that may be used in the emulsions, examples that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof, such as the mixture of glyceryl stearate and PEG-40 stearate.

Mention may also be made of fatty alcohol/alkylpolyglycoside emulsifying mixtures as described in patent applications WO 92/06778, WO 95/13863 and WO 98/47610, for example the commercial products sold by the company SEPPIC under the name Montanov®. Among the emulsifiers that may be used in the compositions according to the invention, examples that may be mentioned include alkyl dimethicone copolyols, for example Cetyl PEG/PPG-10/1 Dimethicone and more particularly the mixture Cetyl PEG/PPG-10/1 Dimethicone and Dimethicone (INCI name), such as the product sold under the brand name Abil EM90 by the company Goldschmidt, or the mixture (Polyglyceryl-4 stearate and Cetyl PEG/PPG-10 (and) Dimethicone (and) Hexyl Laurate), such as the product sold under the brand name Abil WE09 by the same company.

Mention may also be made of dimethicone copolyols, for example PEG-18/PPG-18 Dimethicone and more particularly the mixture Cyclopentasiloxane (and) PEG-18/PPG-18 Dimethicone (INCI name), such as the product sold by the company Dow Corning under the brand name Silicone DC 5225 C or KF-6040 from the company Shin-Etsu.

Mention may also be made of nonionic emulsifiers derived from fatty acids and polyols, alkylpolyglycosides (APGs) and sugar esters, and also mixtures thereof.

As nonionic emulsifiers derived from fatty acids and polyols, use may notably be made of fatty acid esters of polyols, the fatty acid notably containing a C₈-C₂₄ alkyl chain, and the polyols being, for example, glycerol and sorbitan.

The fatty acid esters of polyols that may notably be mentioned include isostearic acid esters of polyols, stearic acid esters of polyols, and mixtures thereof, in particular isostearic acid esters of glycerol and/or of sorbitan.

The stearic acid esters of polyols that may notably be mentioned include polyethylene glycol esters, for instance PEG-30 dipolyhydroxystearate, such as the product sold under the name Arlacel P135 by the company ICI.

The glycerol and/or sorbitan esters that may be mentioned, for example, include polyglyceryl isostearate, such as the product sold under the name Isolan GI 34 by the company Goldschmidt; sorbitan isostearate, such as the product sold under the name Arlacel 987 by the company ICI; sorbitan glyceryl isostearate, such as the product sold under the name Arlacel 986 by the company ICI, the mixture of sorbitan isostearate and polyglyceryl isostearate (3 mol) sold under the name Arlacel 1690 by the company Uniqema, and mixtures thereof.

The emulsifier may also be chosen from alkylpolyglycosides with an HLB of less than 7, for example those represented by the general formula (1) R—O-(G)x (1), in which R represents a branched and/or unsaturated alkyl radical including from 14 to 24 carbon atoms, G represents a reduced sugar including 5 or 6 carbon atoms, and x is a value ranging from 1 to 10 and preferably from 1 to 4, and G notably denotes glucose, fructose or galactose.

The unsaturated alkyl radical may include one or more ethylenically unsaturated groups, and in particular one or two ethylenically unsaturated groups.

Among the alkylpolyglycosides of this type, mention may be made of alkylpolyglucosides (G=glucose in formula (I)), and notably the compounds of formula (I) in which R more particularly represents an oleyl radical (unsaturated C₁₈ radical) or isostearyl (saturated C₁₈ radical), G denotes glucose, x is a value ranging from 1 to 2, notably isostearyl glucoside or oleyl glucoside, and mixtures thereof. This alkylpolyglucoside may be used as a mixture with a coemulsifier, more particularly with a fatty alcohol and notably a fatty alcohol containing the same fatty chain as that of the alkylpolyglucoside, i.e. comprising from 14 to 24 carbon atoms and containing a branched and/or unsaturated chain, for example isostearyl alcohol when the alkylpolyglucoside is isostearyl glucoside, and oleyl alcohol when the alkylpolyglucoside is oleyl glucoside, optionally in the form of a self-emulsifying composition, as described, for example, in WO-A-92/06778. Use may be made, for example, of the mixture of isostearyl glucoside and isostearyl alcohol, sold under the name Montanov WO 18 by the company SEPPIC, and also the mixture of octyldodecanol and octyldodecyl xyloside sold under the name Fludanov 20X by the company SEPPIC.

Mention may also be made of succinic-terminated polyolefins, for instance esterified succinic-terminated polyisobutylenes and salts thereof, notably the diethanolamine salts, such as the products sold under the names Lubrizol 2724, Lubrizol 2722 and Lubrizol 5603 by the company Lubrizol or the commercial product Chemcinnate 2000.

According to a preferred embodiment, the composition according to the invention comprises at least one nonionic emulsifier, preferably chosen from stearic acid esters of polyols and/or of alkylpolyglycosides.

The total amount of emulsifiers in the composition will preferably be, in a composition according to the invention, in active material contents ranging from 0.5% to 8% by weight and more particularly from 1% to 6% by weight relative to the total weight of the composition.

### Base

In embodiments of the electroporation composition, the composition may also comprise at least one base.

The base may be chosen from mineral bases, for instance alkali metal hydroxides, sodium hydroxide, potassium hydroxide, ammonium hydroxides, aqueous ammonia, organic bases, for example monoethanolamine, diethanolamine, triethanolamine, triisopropylamine, tris[(2-hydroxy)-1-propyl]amine, N,N-dimethylethanolamine, 2-amino-2-methyl-1-propanol, 2-amino-2-methyl-1,3-propanediol, triethylamine, dimethylaminopropylamine and amphoteric bases (i.e. bases containing both anionic and cationic functional groups), such as primary, secondary, tertiary or cyclic organic amines, and amino acids. Examples of amphoteric bases that may be mentioned include glycine, lysine, arginine, taurine, histidine, alanine, valine, cysteine, trihydroxymethylaminomethane (TRISTA), triethanolamine, and any mixture thereof.

According to a particular embodiment, the base of the composition is chosen from sodium hydroxide, potassium hydroxide, ammonium hydroxides, ammonia, monoethanolamine, diethanolamine, triethanolamine, tromethamine, and any mixture thereof.

According to a particular embodiment, the base of the composition is sodium hydroxide.

According to a particular embodiment, the base of the composition according to the invention is present in a weight concentration of less than 0.5% by weight, or even less than 0.3% by weight, relative to the total weight of the composition.

Other Additives The cosmetic composition may also comprise at least one additive chosen from the typical adjuvants of the cosmetic field, such as hydrophilic or lipophilic gelling agents, water-soluble or liposoluble active agents, for example anti-ageing active agents, film-forming polymers, preserving agents, sequestrants, antioxidants, solvents, fragrances, odour absorbers, pH correctors, and mixtures thereof.

According to one embodiment, the composition according to the invention may also comprise at least one preserving agent, preferably phenoxyethanol.

A person skilled in the art will take care to select the optional additional adjuvants and/or the amount thereof so that the advantageous properties of the composition are not, or are not substantially, adversely affected by the envisaged addition.

### Physiologically Acceptable Medium

The composition, notably the cosmetic composition, according to the invention comprises a physiologically acceptable medium.

For the purposes of the present invention, the term “physiologically acceptable medium” is intended to refer to a medium that is suitable for the topical administration of a composition.

A physiologically acceptable medium generally has no unpleasant odour or appearance, and is entirely compatible with topical administration. In the present case, when the composition is intended for topical administration, i.e. by application to the surface of the keratin material under consideration, such a medium is notably considered to be physiologically acceptable when it does not cause stinging, tautness or redness that is unacceptable to the user.

In particular, the composition is suited to topical application, i.e. application to the surface of the skin, the scalp and/or the mucous membrane under consideration. Thus, the physiologically acceptable medium is preferably a cosmetically or dermatologically acceptable medium, i.e. a medium that has no unpleasant odour, colour or appearance, and that does not cause the user any unacceptable stinging, tautness or redness.

The composition may then comprise any constituent normally used in the intended application.

Needless to say, a person skilled in the art will take care to select the optional additional compound(s), and/or the amount thereof, such that the advantageous properties of the compounds according to the invention are not, or are not substantially, adversely affected by the envisaged addition.

Unless otherwise indicated, the amounts indicated are expressed as mass percentages.

### Example

To test the effect of the device, a sample of human skin is peripherally attached to a support, with the counterelectrode of the electroporation device placed between the inner face of the skin and the support. The electrode of the electroporation device is placed in contact with the outer face of the skin sample. The case of the electroporation device is immobilized by a support. The support is placed on an orbital shaker, so that the electrode moves over the surface of the skin.

The device used in this example is a BTX Gemini X2 programmable signal generator (Harvard Instruments).

The electroporation treatment time is about 1 minute. It consists of 81 pulses of 10 ms each, with rest periods of 500 ms between the pulses. The polarity chosen is negative, i.e. the electrode is at a negative potential relative to the counterelectrode. On the skin, the voltage between the electrode and the counterelectrode is about 40 V.

50 μl of EGF are applied before the device is placed in position (corresponding to 100 μl/cm²).

EGF is applied here independently of the compound N-acetyl-3-trifluoromethyl-phenyl-(valine-glycine).

The EGF stock solution is prepared by dissolving the synthetic peptide powder in PBS at a concentration of 2.50 pg/ml before starting the experimentation, where 125 ng are applied per cm² of skin explant.

For the compound N-acetyl-3-trifluoromethyl-phenyl-(valine-glycine), 100 μl/cm² of each formulation 1 and 2 are applied before treating the skin with Fusion Meso Xpert, mode S.

The two cosmetic compositions are prepared according to the following protocols: For the preparation of composition 1, 95% (in qs) of deionized water is weighed out. 7% glycerol and 2% dipropylene glycol are added. The mixture is stirred at 350 rpm. 1% of N-acetyl-3-trifluoromethyl-phenyl-(valine-glycine) and 1% of hydroxypropylmethylcellulose are weighed out and then added to the mixture portionwise with stirring to disperse the powder. 0.5% of phenoxyethanol is added. The mixture is stirred at 550 rpm for 15 minutes. The pH is adjusted to 5.5 with sodium hydroxide. The remaining deionized water (qs 100%) is added to reach the desired weight. Composition 1 is of gel type.

For the preparation of composition 2, phase A is mixed and heated to 50° C. to dissolve the active agent. Phase B is added. The fatty phase C is mixed and heated to 30° C. It is then incorporated into the mixture of phases A and B in a single portion. The whole is mixed slowly with a spatula. Diffusion is observed, the whole is mixed until an emulsion is obtained. The whole is mixed for a few minutes using a deflocculator. Phase D is added. Composition 2 is an oil-in-water emulsion.

The compositions have the following formulations (the amounts are expressed on a weight basis, relative to the total weight of the composition):

TABLE 1

| | | Formulation 1 | Formulation 2 |
|---|---|---|---|
| Phases | Compounds (INCI name) | (weight %) | (weight %) |
| A | Water (MICROBIOLOGICALLY | qs 100 | qs 100 |
| | CLEAN DEIONIZED WATER) | | |
| | Sodium Hydroxide (CAUSTIC LYE, GUJ ARAT | 0.24 | 0.11 |
| | ALKALIES AND CHEMICALS) | | |
| | Acetyl Trifluoromethylphenyl ValylGlycine | 1 | 1 |
| | (MEXORYL SAR, CHIMEX) | | |
| | Phenoxyethanol (SEPICIDE LD, SEPPIC) | 0.5 | 0.5 |
| | Dipropylene Glycol (DIPROPYLENE-GLYCOL | 2 | 2 |
| | CARE, BASF) | | |
| | Glycerin (ECOCEROL, ECOGREEN | 7 | 7 |
| | OLEOCHEMICALS) | | |
| | Caprylyl Glycol (HYDROLITE CG ®, SYMRISE) | / | 0.7 |
| B | PEG-30 Dipolyhydroxystearate (CITHROL DPHS- | / | 0.5 |
| | SO-(MV), CRODA) | | |
| | Octyldodecanol (and) Octyldodecyl Xyloside | / | 2 |
| | (FLUIDANOV 20X ® (SEPPIC)) | | |
| C | Cyclohexasiloxane (XIAMETER PMX-0246 | | 6 |
| | CYCLOHEXASILOXANE, DOW CORNING, DOW | | |
| | CHEMICAL) | | |
| | Dimethhicone Crosspolymer (DOWSIL EL-9240 | / | 2 |
| | SILICONE ELASTOMER BLEND, DOW CORNING, | | |
| | DOW CHEMICAL) | | |
| D | Hydroxypropyl Methylcellulose (METHOCEL F 4 | 1 | / |
| | M PERSONAL CARE GRADE) | | |
| | Sodium Acrylate/Sodium Acryloyldimethyl | / | 5 |
| | Taurate Copolymer (and) Isohexadecane (and) | | |
| | Polysorbate 80 (SIMULGEL, SEPPIC) | | |

### Comparative Example

Firstly, compositions 1 and 2 are applied topically, and secondly, their application is combined with electroporation.

Results The skin is removed from the experimental system and then analysed to determine the amount of N-acetyl-3-trifluoromethyl-phenyl-(valine-glycine) which has penetrated, via an ELISA technique.

The results are given in FIG. 4 and in FIG. 5.

It is shown that, by means of the process according to the invention, the amount of N-acetyl-3-trifluoromethyl-phenyl-(valine-glycine) that has penetrated is much higher (log higher in FIG. 5), compared with the topical application of an identical composition.

In the entire description, including the claims, the expression “comprising one” is to be understood as being synonymous with “comprising at least one”, unless the contrary is specified.

The expressions “between . . . and . . . ”, “comprises from . . . to . . . ”, “formed from . . . to . . . ” and “ranging from . . . to . . . ” should be understood as being inclusive of the limits, unless otherwise specified.

Sequence listing

SEQ ID No. 1
NSDSECPLSHDGYCLHDGVCMYIEALDKYACNCVVGYIGERCQYRDLKW
WE

1. Cosmetic and non-therapeutic electroporation process for delivering a composition through human keratin materials, the composition comprising at least one peptide with a molecular weight ranging from 500 Da to 20 kDa, the electroporation process comprising the exposure of the composition in contact with said keratin materials to a succession of pulses of an electric current, the electric current being supplied by an electroporation device having at least one electrode (2) and at least one counterelectrode (3), the pulses each having a duration (tₚᵤₗₛₑ) of between 1 ms and 5 s, with a rest interval (Toff) between them of between 0.1 s and 5 s, and the electric current having a voltage amplitude ranging from 20 V to 400 V, and a current ranging from 0.1 mA to 10 mA.

2. The process of claim 1, the pulse duration (tₚᵤₗₛₑ) being between 5 ms and 3 s.

3. The process of claim 1, the rest interval (Toff) being between 0.3 s and 3 s.

4. The process of claim 1, the voltage amplitude ranging from 25 V to 300 V.

5. The process of claim 1, the current ranging from 0.2 mA to 2 mA.

6. The process of claim 1, the number N of consecutive pulses applied being between 2 and 15 000.

7. The process of claim 1, the applied current having a duty cycle (tₚᵤₗₛₑ/(tₚᵤₗₛₑ+T_{off})) ranging from 10% to 90%, where tₚᵤₗₛₑ denotes the duration of a pulse and T_{off} denotes the interval between two successive pulses.

8. The process of claim 1, the electric current having a direct polarity.

9. The process of claim 1, the surface area of the electrode(s) (2) in contact with the keratin materials ranging from 2 to 20 cm².

10. The process of claim 1, the counterelectrode being static and having a surface area in contact with the human keratin materials that is greater than that of the electrode, which is moved in contact with the keratin materials.

11. The process of claim 1, the maximum voltage between the electrode (2) and the counterelectrode (3) in open circuit ranging from 150 V to 250 V.

12. The process of claim 1, consisting in manually moving the electrode(s) (2) of the electroporation device over the keratin materials while subjecting said keratin materials to said pulses of electric current.

13. The process of claim 1, the electric current having a square or rectangular waveform.

14. The process of claim 1, the electroporation device (1) being hand-held.

15. The process of claim 1, comprising topical application of the composition to the zone to be treated before applying the electric current and/or during said application.

16. The process of claim 1, the electrode(s) (2) having a rounded surface for contact with the keratin materials.

17. The process of claim 1, wherein the peptide with a molecular weight ranging from 500 Da to 20 kDa is chosen from elastase-inhibiting N-acylamino amide compounds.

18. The process of claim 1, wherein the peptide with a molecular weight ranging from 500 Da to 20 kDa is chosen from the elastase-inhibiting N-acylamino amide compounds of formula (I): 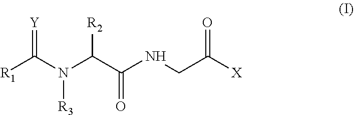
in which:
the radical Y represents O or S;
the radical R₁ represents:
(i) a hydrogen atom;
(ii) a linear, branched or cyclic, saturated or unsaturated hydrocarbon-based radical containing from 1 to 18 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; and —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; —C-Hal₃ (halogen); with R″ representing a linear, branched or cyclic, saturated or unsaturated hydrocarbon-based radical containing 1 to 6 carbon atoms, which is optionally halogenated, or even perhalogenated;
(iii) a radical chosen from the radicals —OR; —NH₂; —NHR; —NRR′; —NH—COR; —COOR; —COR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₂ represents a linear, branched or cyclic, saturated or unsaturated hydrocarbon-based radical containing 1 to 18 carbon atoms, optionally substituted with 1 to 5 identical or different groups chosen from —OH; —OR; —O—COR; —SH; —SR; —S—COR; —NH₂; —NHR; —NRR″; —NH—COR; -Hal (halogen); —CN; —COOR; and —COR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical R₃ represents a radical chosen from those of formula (II) or (III):
-A-C₆H_{(5-y)}—B_{y} (II)
—C₆H_{(5-y)}—B_{y′} (III)
in which:
y is an integer between 0 and 5 inclusive;
y′ is an integer between 0 and 5 inclusive;
A is a divalent, linear, branched or cyclic, saturated or unsaturated, hydrocarbon-based radical containing 1 to 18 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; and —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
B is a divalent, linear, branched or cyclic, saturated or unsaturated hydrocarbon-based radical containing 1 to 18 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen, or even perhalogen); —C-Hal₃ (halogen); —CN; —COOR; —COR; —NO₂; and —SO₂—OR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
the radical X represents a radical chosen from —OH; —OR₄; —NH₂; —NHR₄; —NR₄R₅; —SR₄; —COOR₄; and —COR₄;
with R₄ and R₅ representing, independently of each other, a linear, branched or cyclic, saturated or unsaturated hydrocarbon-based radical containing 1 to 6 carbon atoms, optionally substituted with 1 to 5 identical or different groups chosen from —OH; —OR; —O—COR; —SH; —SR; —S—COR; —NH₂; —NHR; —NRR′; —NH—COR; -Hal (halogen, or even perhalogen); —CN; —COOR; and —COR;
with R and R′ representing, independently of each other, a linear, branched or cyclic, saturated or unsaturated hydrocarbon-based radical containing 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
it being possible for said radicals R₄ and R₅ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical containing 1 to 6 carbon atoms, which is optionally halogenated, or even perhalogenated;
mineral or organic acid salts thereof, optical isomers thereof, in isolated form or as a racemic mixture.

19. The process of claim 1, wherein the peptide with a molecular weight ranging from 500 Da to 20 kDa is at least N-acetyl-3-trifluoromethyl-phenyl-(valine-glycine).

20. The process of claim 1, wherein the peptide with a molecular weight ranging from 500 Da to 20 kDa is at least the epidermal growth factor EGF of sequence SEQ ID No. 1.

21. The process of claim 1, wherein the composition comprises at least one peptide with a molecular weight ranging from 500 Da to 20 kDa, in a content of between 0.2% and 10% by weight, relative to the total weight of the composition.

22. The process of claim 1, wherein the composition comprises 1% by weight of at least one peptide with a molecular weight ranging from 500 Da to 20 kDa.

23. The process of claim 1, wherein the composition has a water content of between 20% and 100% by weight, relative to the total weight of the composition.

24. The process of claim 1, wherein the composition comprises at least one alcohol.

25. The process of claim 1, wherein the composition comprises a thickener/gelling agent.

26. The process of claim 1, wherein the composition is of gel type, or is an oil-in-water emulsion.

27. The process of claim 1, wherein the composition has a pH ranging from 2 to 7.5.

28. Kit for performing the process of claim 1, including:
an electroporation device containing at least one electrode (2) and at least one counterelectrode (3) and arranged to subject the keratin materials to a succession of pulses of an electric current, the pulses each having a duration (tₚᵤₗₛₑ) of between 1 ms and 5 s, with a rest interval (T_{off}) between them of between 0.1 s and 5 s, and the electric current having a voltage amplitude ranging from 20 V to 400 V, and a current ranging from 0.1 mA to 10 mA, and
a composition comprising at least one peptide with a molecular weight ranging from 500 Da to 20 kDa.

29. The process of claim 1, wherein the electric current having a voltage amplitude ranges from 25 V to 300 V.

30. The process of claim 8, the polarity of the electrode being negative relative to that of the counterelectrode.

31. The process of claim 10, the surface area of the counterelectrode being more than 1.5 times that of the electrode.
